(19) United States
(12) Patent Application Publication
(10) Pub. No.: US 2025/0221677 A1
(43) Pub. Date: Jul. 10, 2025

(54) TEST PHANTOM FOR EVALUATING ELECTROMAGNETIC RADIATION IMAGES

(71) Applicant: KAOHSIUNG MEDICAL UNIVERSITY, Kaohsiung (TW)

(72) Inventor: Ming-Chung Chou, Kaohsiung (TW)

(21) Appl. No.: 19/010,213

(22) Filed: Jan. 6, 2025

Related U.S. Application Data
(60) Provisional application No. 63/618,408, filed on Jan. 8, 2024.

(51) Int. Cl. A61B 6/00; A61B 6/58

(52) U.S. Cl. CPC A61B 6/44; A61B 6/583

(57) ABSTRACT

A test phantom for evaluating electromagnetic radiation images is adapted for solving the problem of high requirement of manufacturing precision for detail evaluation portions formed by concave portions in the conventional phantom. The test phantom includes a plurality of layer bodies and a plurality of recess portions. The plurality of layer bodies is stacked one upon another to form stacked layer bodies. An area of each of the plurality of layer bodies gradually increases from top to bottom of the stacked layer bodies, and each of the plurality of layer bodies has an exposed region on an upper surface thereof. The plurality of recess portions is formed by recessing the exposed regions of the upper surfaces of at least two of the plurality of layer bodies. The recess portions located on different layer bodies cause different degrees of attenuation to electromagnetic radiation passing therethrough.

### CROSS REFERENCE TO RELATED APPLICATION

The application claims the benefit of U.S. provisional application Ser. No. 63/618,408, filed on Jan. 8, 2024, and the entire contents of which are incorporated herein by reference.

### BACKGROUND OF THE INVENTION

### 1. Field of the Invention

The present invention relates to a test phantom and, more particularly, to a test phantom for evaluating electromagnetic radiation images.

### 2. Description of the Related Art

As shown in FIG. 1, a first conventional phantom 6 specifically designed for assessing image contrast resolution is depicted, which is exemplified by the MTF phantom from Leeds Test Objects Ltd., where “MTF” stands for “modulation transfer function”. The phantom 6 includes a plate 61 and a shielding member 62 that is rotatably connected to the plate 61, with multiple scale markers M displayed on the plate 61. During use, the phantom 6 evaluates images generated at the emission center of an electromagnetic radiation instrument. The evaluation is conducted by manually rotating the shielding member 62 to align with different scale markers M, enabling corresponding image assessment. However, the first conventional phantom 6 is limited to a small area in the center of the generated image, which restricts its effectiveness in evaluating the contrast resolution at the image periphery. Consequently, the measured contrast resolution becomes distorted, with resolution decreasing as the evaluation moves further from the emission center. Additionally, frequent adjustments of the shielding member 62 during the measurement process lead to inconvenience in measurement and assessment.

As shown in FIG. 2, a second conventional phantom 7 is exemplified by the TO16 phantom from Leeds Test Objects Ltd. The second phantom 7 features multiple concave portions 7C and corresponding fillers 7F that form detail evaluation portions. The second phantom 7 further includes a ring 7R at the outer periphery thereof. While the detail evaluation portions (particularly the fillers 7F) are used for assessing image detail resolution, the ring 7R serves only as a protective component of the fillers and is not intended for contrast resolution evaluation. Specifically, the multiple concave portions 7C are categorized into several detail groups. Within each group, the concave portions 7C and fillers 7F have identical diameters but differ in density, whereas the diameters of the concave portions 7C and fillers 7F differ across groups. Thus, the detail resolution can be evaluated from the images generated by the multiple fillers 7F with different diameters and densities. However, although the overall body of the phantom 7 is configured in a radially symmetrical arrangement around the central point (circle center), since the configuration of the multiple fillers 7F are not symmetrically arranged or not distributed in a ring array, it fails to evaluate the corresponding detail resolution under uneven directional electromagnetic radiation conditions. Furthermore, the requirements for the fillers 7F with varying densities and for the detail groups with varying diameters increase the demand in manufacturing precision. Additionally, as the ring 7R is made of non-metallic materials, it is incapable of accurately assessing the corresponding contrast resolution under conditions of directional non-uniformity in electromagnetic radiation based on the same standard.

As shown in FIG. 3, a third conventional phantom 8 is exemplified by CDRAD phantom produced by Artinis Medical Systems. The phantom 8 includes a plurality of concave portions 8C used for assessing image detail resolution. These concave portions 8C are arranged with aperture size varying along the y-axis and depth varying along the x-axis. However, since the overall body and the plurality of concave portions 8C of the conventional phantom 8 are neither symmetrically arranged around a central point thereof nor distributed in a ring array, it fails to evaluate detail resolution effectively under uneven directional electromagnetic radiation conditions. Moreover, the varying depths required for the plurality of concave portions 8C further increase the demand in manufacturing precision.

As shown in FIG. 4, a fourth conventional phantom 9 is disclosed in Taiwan Patent No. I677323. The phantom 9 is formed by stacking multiple circular layer bodies 9L. Since the phantom 9 is symmetrically arranged around a central point in the projection plane of electromagnetic radiation, it can utilize image information generated from electromagnetic radiation projections to evaluate image uniformity quantitatively by using mutual information (MI). However, the phantom 9 is less sensitive in assessing contrast resolution or detail resolution.

According to the above, although the third conventional phantom 8 eliminates the need for fillers 7F of varying densities and sizes as required in the second conventional phantom 7, the concave portions 8C of the third conventional phantom 8 must be arranged on the same layer body with varying depths, leading to higher manufacturing precision requirements and increased manufacturing costs. Furthermore, because the features for image quality evaluation in the first through third conventional phantoms 6-8 are neither symmetrically arranged nor distributed in a ring array, the placement direction of the first through third conventional phantoms 6-8 can significantly impact image quality evaluation results due to the uneven radiation effect caused by the heel effect in electromagnetic radiation (especially X-rays). Additionally, to effectively evaluate multiple aspects such as contrast resolution, detail resolution and uniformity, it is necessary to use and switch between different phantoms. The process of replacing phantoms can cause deviations from the reference center in the previous measurement, while variations in radiation output between successive X-ray exposures can further complicate the stable and effective evaluation of overall image quality.

In light of the above, it is necessary to improve the conventional test phantoms.

### SUMMARY OF THE INVENTION

To solve the above problems, it is an objective of the present invention to provide a test phantom for evaluating electromagnetic radiation images which simplifies the manufacturing process of recess portions/detailed evaluation portions.

It is another objective of the present invention to provide a test phantom for evaluating electromagnetic radiation images which enables simultaneous assessment of multidirectional quality indicators.

It is a further objective of the present invention to provide a test phantom for evaluating electromagnetic radiation images which enables simultaneous testing of various image quality indicators.

As used herein, the term “electromagnetic radiation” is a generic term encompassing various forms of light waves or electromagnetic waves. Thus, as along as the “electromagnetic radiation” can generate a corresponding image to form an “electromagnetic radiation image”, it is suitable for evaluation image quality using the test phantom in the present invention. In other words, the test phantom of the present invention is applicable not only to the evaluation of X-ray image quality but also to the quality assessment of images produced by other forms of non-visible light, visible light, or electromagnetic waves.

As used herein, the term “engagement”, “coupling”, “assembly”, “arrangement”, “stack” or similar terms is used to include separation of connected members without destroying the members after connection or inseparable connection of the members after connection. A person having ordinary skill in the art would be able to select according to desired demands in the material or assembly of the members to be connected.

A test phantom for evaluating electromagnetic radiation images according to the present invention includes a plurality of layer bodies and a plurality of recess portions. The plurality of layer bodies is stacked one upon another to form stacked layer bodies. An area of each of the plurality of layer bodies gradually increases from top to bottom of the stacked layer bodies, and each of the plurality of layer bodies has an exposed region on an upper surface thereof. The plurality of recess portions is formed by recessing the exposed regions of the upper surfaces of at least two of the plurality of layer bodies. The recess portions located on different layer bodies cause different degrees of attenuation to electromagnetic radiation passing therethrough.

Thus, the test phantom for evaluating electromagnetic radiation images according to the present invention can achieve the effect of evaluating detailed contrast characteristics of images based on the thickness of the corresponding layer bodies to be passed through by the electromagnetic radiation, especially in all cases where the recess portions have the same depth (including through-holes) or different depths.

In an example, the test phantom has a centroid in a viewing direction, and the plurality of recess portions is symmetrically arranged or is distributed in a ring array around the centroid in the viewing direction. Thus, uniformity of images in all directions can be evaluated.

In an example, after stacking, each of the plurality of layer bodies renders an outline of the test phantom to be symmetrically arranged or to be distributed in a ring array around a centroid of the test phantom in a viewing direction. Thus, uniformity of images in all directions can be evaluated.

In an example, in each two adjacent layer bodies, with each of which having at least one of the plurality of recess portions, a radial length of one or more of the at least one of the plurality of recess portions in a lower one of the two adjacent layer bodies is not less than a radial length of any of the at least one of the plurality of recess portions in an upper one of the two adjacent layer bodies. Thus, detailed contrast characteristics of images can be evaluated.

In an example, in each two adjacent layer bodies, with each of which having at least one of the plurality of recess portions, a radial length of one or more of the at least one of the plurality of recess portions in a lower one of the two adjacent layer bodies is greater than a radial length of any of the at least one of the plurality of recess portions in an upper one of the two adjacent layer bodies. Thus, by making the radial length of an upper layer body greater than that of a lower layer body, together with the feature that an upper layer body causes a greater degree of attenuation of the electromagnetic radiation, detailed contrast characteristics of images can be more precisely evaluated based on the relationship between the thickness and the radial length.

In an example, each of the plurality of recess portions is a through-hole. Thus, the manufacture of the plurality of recess portions of the plurality of layer bodies can be simplified. Furthermore, detailed contrast characteristics of images can still be evaluated based on the thickness of the corresponding layer bodies to be passed through by the electromagnetic radiation.

In an example, one of the plurality of layer bodies has a plurality of recess portions with different radial lengths, and the radial lengths are arranged according to the Fibonacci Sequence. Thus, detailed contrast characteristics of images can be effectively evaluated by selecting a recess portion with an appropriate radial length.

In an example, a bottommost layer body in the plurality of layer bodies has a metal ring disposed around an outer periphery thereof. Thus, contrast resolution of images can be evaluated through the arrangement of the metal ring.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a perspective view of the first conventional phantom.

FIG. 2 is a schematic diagram showing the configuration of the second conventional phantom.

FIG. 3 is a schematic diagram showing the configuration of the third conventional phantom.

FIG. 4 shows top and front views of the structure of the fourth conventional phantom.

FIG. 5 is a schematic diagram showing the test phantom of the present invention in use.

FIG. 6 shows top and front views of the structure of the first preferred embodiment of the test phantom according to the present invention.

FIG. 7 is a top view of the structure of the second preferred embodiment of the test phantom according to the present invention.

FIG. 8 is an exploded, perspective view illustrating the assembly of the layer bodies of the test phantom.

FIG. 9 shows an image acquired through electromagnetic radiation exposure of the test phantom as shown in FIG. 7.

FIG. 10 shows an example using the image from FIG. 9 to assess image uniformity in all orientations for the zones of interest.

FIG. 11 shows the image uniformity evaluation of a single layer body in all orientations in the zones of interest from the image in FIG. 10.

FIG. 12 shows an example of evaluating image contrast resolution at the edge zones of the metal ring and background using the image from FIG. 9.

FIG. 13 shows an example of evaluating image detail resolution for the recess portions using the image from FIG. 9.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

### DETAILED DESCRIPTION OF THE INVENTION

In order to make the above and other objectives, features, and advantages of the present invention clearer and easier to understand, preferred embodiments of the present invention will be described hereinafter in connection with the accompanying drawings. Furthermore, the elements designated by the same reference numeral in various figures will be deemed as identical, and the description thereof will be omitted.

Please refer to FIG. 5, which shows a front view of the of the test phantom for evaluating electromagnetic radiation images in use. The test phantom 1 is placed on an imaging platform IP and an emitting device ED, such as an X-ray emitter, is used to project corresponding electromagnetic radiation (e.g., X-ray) to generate an image shielded by the test phantom 1 on a sensing board SB. The image is used to evaluate the imaging quality of the emitting device ED or the characteristics of the electromagnetic radiation source.

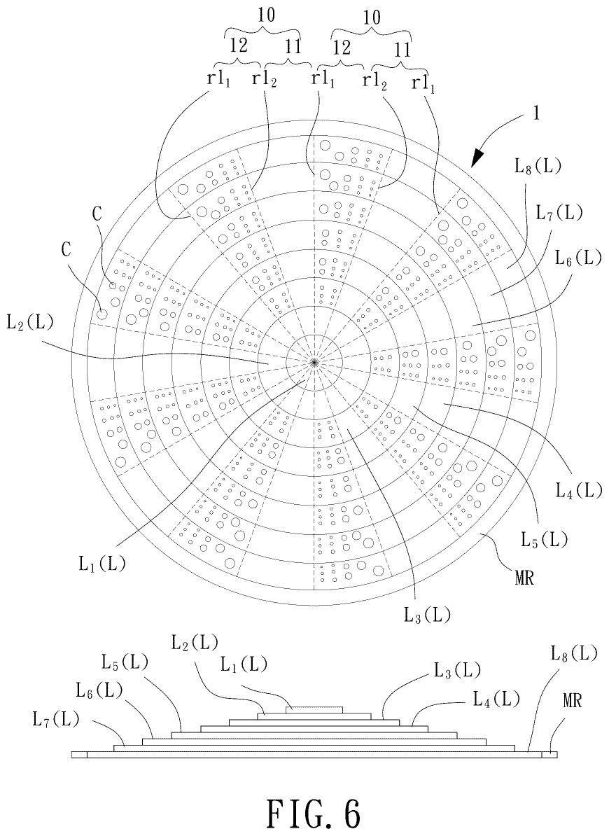

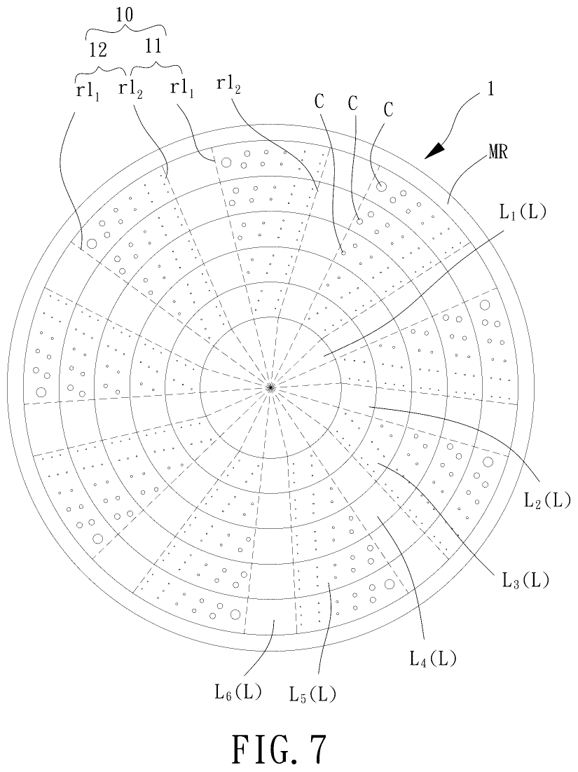

Referring to FIGS. 6 and 7, FIG. 6 shows the first embodiment of the test phantom for evaluating electromagnetic radiation images, and FIG. 7 shows the second embodiment of the test phantom for evaluating electromagnetic radiation images. Both embodiments are based on the same inventive concept with certain differences in arrangement, particularly in the number and specific arrangement of layer bodies L and recess portions C. The test phantom 1 of the present invention includes a plurality of layer bodies L, a plurality of recess portions C, and optionally a metal ring MR. The plurality of recess portions C are formed by recessing a surface of the plurality of layer bodies L, and the metal ring MR is disposed circumferentially around an outer periphery of the bottommost layer body L in the plurality of layer bodies L. Notably, corresponding to the configuration of the plurality of layer bodies L and the plurality of recess portions C, a plurality of test areas 10 can be defined on the test phantom 1, each preferably provided with a first subarea 11 and a second subarea 12.

The number of the plurality of layer bodies L can be defined as N (where N is any positive integer not less than 2), and can be designated from one end to the other in a viewing direction as layer bodies L₁ to L_{N}. In other words, the test phantom 1 includes stacked layer bodies L₁ to L_{N}. In a top view for example, the N layer bodies from the topmost to the bottommost are designated as L₁ to L_{N}, with N being 7 in FIG. 6 (namely, having seven layers) and with N being 6 in FIG. 7 (namely, having six layers). An area of each of the plurality of layer bodies L₁ to L_{N} forming the stack increase from top to bottom, and each of the plurality of layer bodies L₁ to L_{N} has an exposed region on an upper surface thereof. Preferably, the top surface of each of the plurality of layer bodies L₁ to L_{N} is a flat surface. Since electromagnetic radiation images may require sensing boards SB of various sizes (as shown in FIG. 5) according to actual clinical needs, the test phantom 1 can be formed by layer bodies L with different numbers, shapes, and sizes to cover the entire sensing board SB.

In terms of shape, the plurality of layer bodies L can have any shape, preferably circular. Particularly, in a viewing direction, the test phantom 1 has a centroid, and the plurality of layer bodies L is stacked in alignment with their respective centroids in the viewing direction. Preferably, the outline of the test phantom 1 presents a symmetric arrangement or a ring array distribution about the centroid in the viewing direction.

Particularly, each of the plurality of layer bodies L₁ to L_{N} has a certain thickness, which can range from 0.5 to 20 mm, preferably 2 mm. The present invention is not limited in this regard and the thickness of the layer body L can be adjusted in an unit of 0.01 mm. Optionally, the thickness of the each of the plurality of layer bodies L₁ to L_{N} may be identical, partially identical, or entirely different.

Optionally, each of the plurality of layer bodies L₁ to L_{N} can be made from any non-metallic material, preferably polymethyl methacrylate (acrylic). Specifically, in cases that the electromagnetic radiation used can penetrate non-transparent materials, the test phantom 1 can be formed by layer bodies L with any transparency. Conversely, in cases that the electromagnetic radiation used cannot penetrate non-transparent materials, the test phantom 1 can be formed by transparent or semi-transparent layer bodies L. Preferably, “transparent” refers to materials with a transmittance of at least 80%, while “semi-transparent” refers to materials with a transmittance of 20-80%.

Optionally, when each of the plurality of layer bodies LI to LN is circular and stacked concentrically, the total lengths of the exposed regions of each of the multiple layer bodies L₁ to L_{N} in the radial direction may be all equal, partially equal, or all unequal. For example, in the examples shown in FIG. 6 or 7, in the top view, the total lengths of the exposed regions of each of the multiple layer bodies L₁ to L_{N} in the radial direction are all equal and symmetrically configured with respect to the centroid. In another example (not shown), the areas of the exposed regions of each of the multiple layer bodies L₁ to L_{N} are equal, resulting in the total lengths of each exposed region in the radial direction being all unequal.

Each recess portion C is formed by recessing a surface (particularly an upper surface) of the corresponding layer body L. The depths of each recess portion C may be the same or different. Preferably, the depths of each recess portion C may be the same to facilitate manufacturing. More preferably, each recess portion C forms a through-hole, which is more advantageous for manufacturing.

Optionally, each layer body L can be configured with or without the recess portion C according to actual needs. In particular, some of the plurality of layer bodies L may not have the recess portion C, and it is preferable that at least two layer bodies L have the recess portion C. As shown in the example of FIG. 6, the topmost and second topmost layer bodies L₁ and L₂ are not provided with the recess portion C. As depicted in the example of FIG. 7, the topmost layer body L1 is not provided with the recess portion C. Optionally, in another example (not shown), all layer bodies L can be provided with at least one recess portion C. Optionally, in yet another example (not shown), the recess portion C may be provided on two layer bodies L with at least one layer body L having no recess portion C in between.

Optionally, from a viewing direction, the plurality of recess portions C is symmetrically configured or arranged in a ring array with respect to the centroid of the whole test phantom 1. Thus, through the configuration of the plurality of recess portions C as described above, the image quality of electromagnetic radiation in various orientations can be tested based on the same configuration of the same standard.

Especially, in a certain viewing direction, the plurality of recess portions C has various radial lengths, and the radial lengths of these recess portions C can be adjusted according to the needs for inspecting the imaging quality. Optionally, in an example, for two adjacent layer bodies L each having at least one recess portion C, the radial length of one or more of the at least one recess portion C in the lower layer body L is not smaller than the radial length of any of the at least one recess portion C in the upper layer body L.

Based on the above relationship regarding the presence or absence of the recess portion C in each layer body L and the configuration of the radial lengths, taking FIG. 6 as an example, the first and second layer bodies L1 and L2 may optionally not have the recess portion C. The third layer body L3 has six recess portions C with different radial lengths. The fourth layer body L4 has seven recess portions C with different radial lengths, six of which can be the same as those in the third layer body L3, and one of which has a radial length greater than each of those in the third layer body L3. The fifth layer body L5 has seven recess portions C with different radial lengths, all seven of which can be the same as those in the fourth layer body L4. The sixth layer body L6 has eight recess portions C with different radial lengths, seven of which can be the same as those in the fifth layer body L5, and one of which has a radial length greater than each of those in the fifth layer body L5. The seventh layer body L7 has nine recess portions C with different radial lengths, eight of which can be the same as those in the sixth layer body L6, and one of which has a radial length greater than each of those in the sixth layer body L6. The eighth layer body L8 has nine recess portions C with different radial lengths, all of which can be consistent with those in the seventh layer body L7.

Taking FIG. 7 as an example, the first layer body L1 may optionally not have the recess portion C. The second layer body L2 has six recess portion C, three of which each have a first radial length, and the other three each have a second radial length, with the second radial length being greater than the first radial length. In other words, there are two types of recess portions C with varying radial lengths. The third layer body L3 has eight recess portions C, compared to the second layer body L2, the third layer body L3 has two additional recess portions C each having a third radial length, and the third radial length is greater than the second radial length. In other words, there are three different types of recess portions C with varying radial lengths. The fourth layer body L4 has ten recess portions C, which, compared to the third layer body L3, has two additional recess portions C each with a fourth radial length, and the fourth radial length is greater than the third radial length. In other words, there are four different types of recess portions C with varying radial lengths. The fifth layer body has twelve recess portions C, which, compared to the fourth layer body L4, has two additional recess portions C each with a fifth radial length, and the fifth radial length is greater than the fourth radial length. In other words, there are five different types of recess portions C with varying radial lengths. The sixth layer body L6 has thirteen recess portion C, which, compared to the layer body L5 of the fifth layer, has one additional recess portion C with a sixth radial length, and the sixth radial length is greater than the fifth radial length. In other words, there are six different types of recess portions C with varying radial lengths.

It should be noted that in the embodiments shown in FIGS. 6 and 7, the corresponding test phantoms 1 both have some layer bodies L without the recess portion C, but the configuration of recess portion C in the present invention is not limited thereto. For example, in other configuration aspects, each layer body L is provided with the recess portion C. Preferably, in the plurality of layer bodies L₁ to L_{N}, the lower layer body Lᵢ₊₁ has at least one recess portion C with a diameter greater than the recess portion C of the upper layer body Lᵢ, where i is a positive integer from 1 to N−1. This can achieve a more accurate assessment of the detail contrast characteristics of electromagnetic radiation images based on the relationship between thickness and radial length.

Optionally, the radial lengths of each recess portion C can be generated in ascending order according to the Fibonacci sequence. For instance, assuming the smallest radial length is 0.1 mm, the radial lengths of the recess portions C on a single layer body L with five different radial lengths can be configured as 0.1 mm, 0.2 mm, 0.3 mm, 0.5 mm, and 0.8 mm. In particular, when the number of recess portions C in the upper layer body L is the same as that in the lower layer body L, the configuration of the radial lengths of the recess portions C is identical. For instance, if the upper layer body L has two recess portions C with radial lengths of 0.1 mm and 0.2 mm, respectively, the lower layer body L also has two recess portions C with radial lengths of 0.1 mm and 0.2 mm, respectively. In particular, when the lower layer body L has more recess portions C than the upper layer body L, the additional recess portions C in the lower layer body L are each larger in radial length than every recess portions C in the upper layer body L.

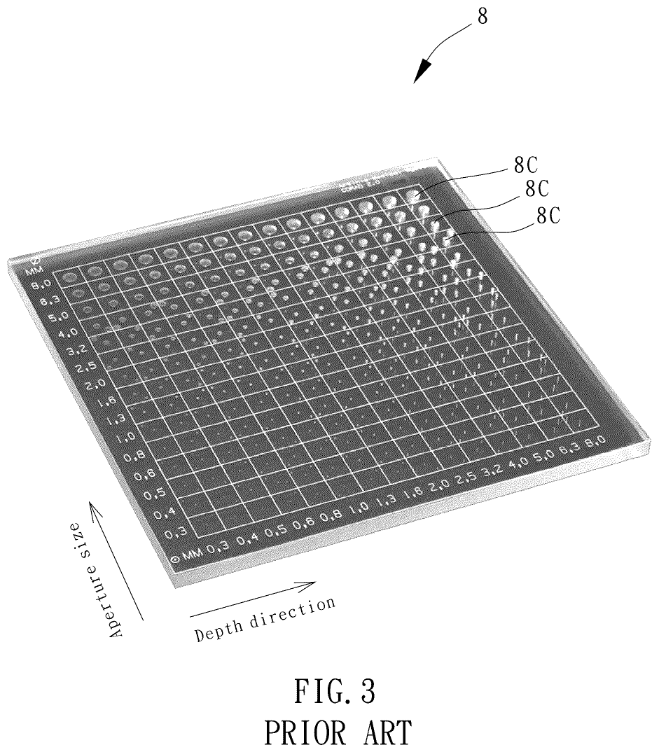

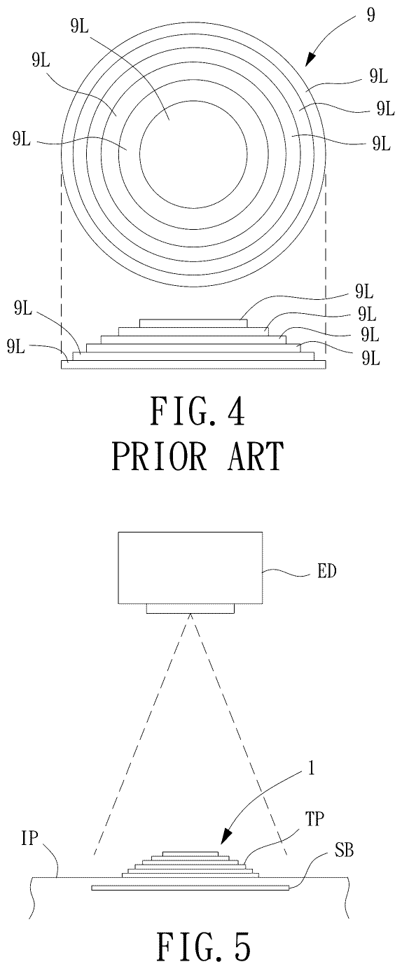

It is particularly noteworthy that, due to the upper layer body Lᵢ having a greater thickness compared to the lower layer body Lᵢ₊₁, even if the depths of the recess portion C in each of the layer bodies L₁ to L_{N} are the same, the energy attenuation of electromagnetic radiation passing through the corresponding recess portions C will be directly proportional to the thickness of the corresponding layer body L. This can produce an image effect similar to that of the third conventional phantom 8 (as shown in FIG. 3), where various concave portions 8C with different depths are configured on a single layer body. Furthermore, by the upper layer body Lᵢ having recess portion C with a smaller aperture than the lower layer body Lᵢ₊₁, it is possible to assess, based on the increased energy attenuation of electromagnetic radiation passing through the recess portion C in the upper layer body Lᵢ, whether the resulting image still manifests the recess portion C with the smaller aperture. This can achieve the effect of distinguishing whether the electromagnetic radiation can produce finer detail resolution.

According to the above, in a specific viewing direction, the plurality of recess portions C is symmetrically configured or arranged in a ring array with respect to the centroid of the whole test phantom 1, corresponding multiple test areas 10 can be defined on the test phantom 1/each layer body L to facilitate the illustration of the functions of using the test phantom 1 in various image quality tests. Using the centroid of the test phantom 1 as a reference point, the test phantom 1 is divided into several test areas 10 of the same shape on the upper surface of each layer body L in the radial direction according to a specific angle interval. Within each of the test area 10, a first subarea 11 and a second subarea 12 are provided, which are alternately arranged circumferentially. In particular, the first subareas 11 and the second subareas 12 are configured to alternate around the centroid of the test phantom 1. Optionally, the test area 10, the first subarea 11, and the second subarea 12 are symmetrically arranged about the centroid of the test phantom 1 or distributed in a ring array. Each first subarea 11 is a region including flat upper surface areas of the plurality of layer bodies L; and in each second subarea 12, some (at least two) or all of the plurality of layer bodies L each have at least one recess portion C.

In particular, regarding the configuration of the specific angle, taking FIGS. 6 and 7 as examples, the specific angle is 40 degrees, and nine test areas 10 are defined. However, the specific angle/number of test areas in the present invention is not limited in this regard, and at least two test areas 10 can be provided according to actual needs, preferably four or more. The number of test areas 10 can be adjusted by increasing or decreasing one test area 10 gradually.

To elaborate on the test area 10, reference can be made to the first reference lines rl₁ and the second reference lines rl₂ as shown in FIGS. 6 and 7. In particular, the first reference lines rl₁ are located on the counterclockwise sides of the second subareas 12 (or on the clockwise sides of the first subareas 11), and the second reference lines rl₂ are located on the clockwise sides of the second subareas 12 (or on the counterclockwise sides of the first subareas 11). Thus, the test area 10 can be defined by two adjacent first reference lines rl₁ (or two adjacent second reference lines rl₂). The first subarea 11 can be defined by the sides of the first reference lines rl₁ in the counterclockwise direction and the sides of the second reference lines rl₂ in the clockwise direction. The second subarea 12 can be defined by the sides of the first reference lines rl₁ in the clockwise direction and the sides of the second reference lines rl₂ in the counterclockwise direction. It should be noted that the first reference lines rl₁ and the second reference lines rl₂ are not limited to straight lines, and can also be any straight lines, arcs, curves or combinations of various line segments.

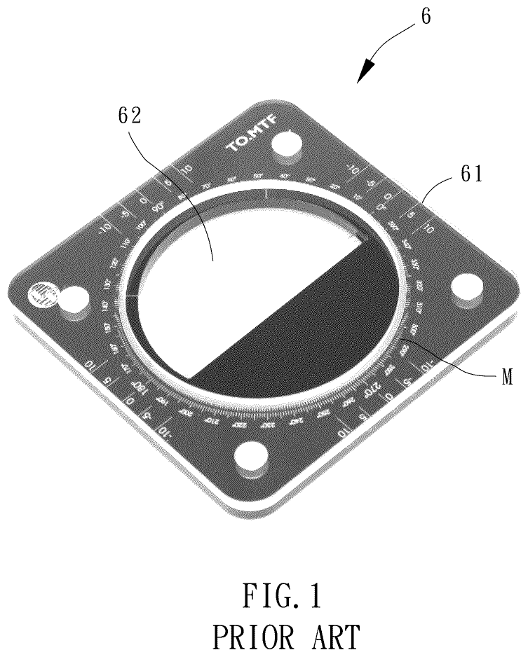

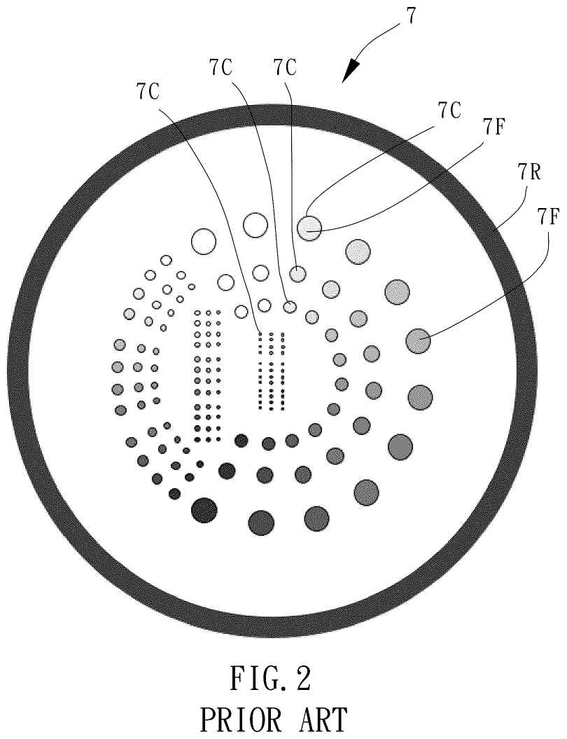

Optionally, the bottommost layer body L_{N} is provided with a metal ring MR disposed around an outer periphery thereof, and the metal ring MR has a flat upper surface. Thus, by providing the metal ring MR, the MTF values at various angles can be obtained to evaluate the contrast resolution in different angular directions. In particular, compared to the first conventional phantom 6 in FIG. 1, which can only detect contrast resolution near the corresponding phantom center, the contrast resolution calculated by the metal ring MR disposed around the outer periphery of the bottommost layer body L_{N} is of greater reference value. In particular, the metal ring MR can be made of a material with a high X-ray attenuation coefficient, preferably tungsten.

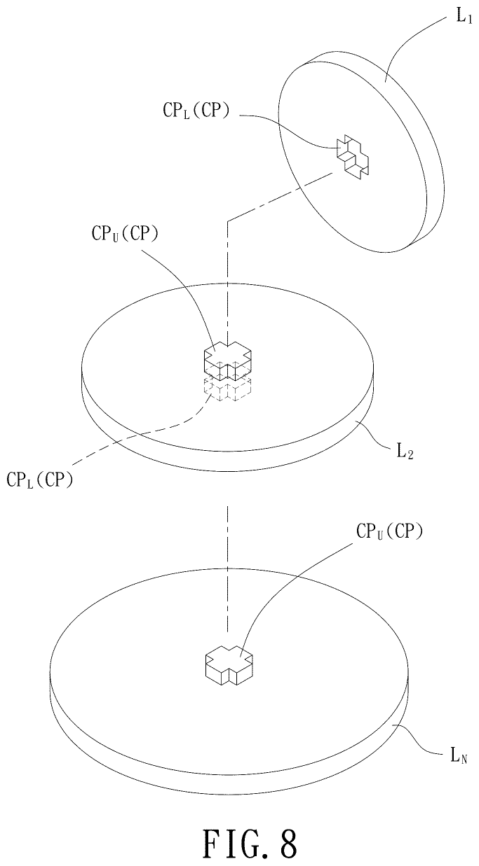

Please refer to FIG. 8, which shows coupling portions CP by which that the layer bodies L of the test phantom 1 in FIGS. 6 and 7 can be aligned and stacked. Specifically, the coupling portion CP can facilitate alignment by providing corresponding concave and convex structures on two adjacent layer bodies L. In the example shown in FIG. 8, a lower surface of the topmost layer body L₁ has a lower coupling portion CP_{L}, and the upper surface of the bottommost layer body L_{N} has an upper coupling portion CP_{U}. Each of the intermediate layer bodies L₂ to L_{N-1} (with only the second layer body L2 schematically shown in FIG. 8) has an upper coupling portion CP_{U} and a lower coupling portion CP_{L} on the upper and lower surfaces, respectively. In two adjacent layer bodies L, the lower coupling portion CP_{L} of the upper layer body Lᵢ is coupled with the upper coupling portion CP_{U} of the lower layer body Lᵢ₊₁. It should be noted that when one in the coupling pair of upper coupling portion CP_{U} and lower coupling portion CP_{L} includes a protruding structure, the other in the coupling pair of the upper coupling portion CP_{U} and lower coupling portion CP_{L} includes a concave structure that matches the protruding structure in the shape.

Based on the structure configuration of the test phantom 1 described above, particularly the arrangement of each layer body L in the test phantom 1 shown in FIG. 7, various structural features of the test phantom 1 can be utilized to obtain various image quality indicators for the images generated by the electromagnetic radiation to be evaluated. Especially, the electromagnetic radiation is X-ray.

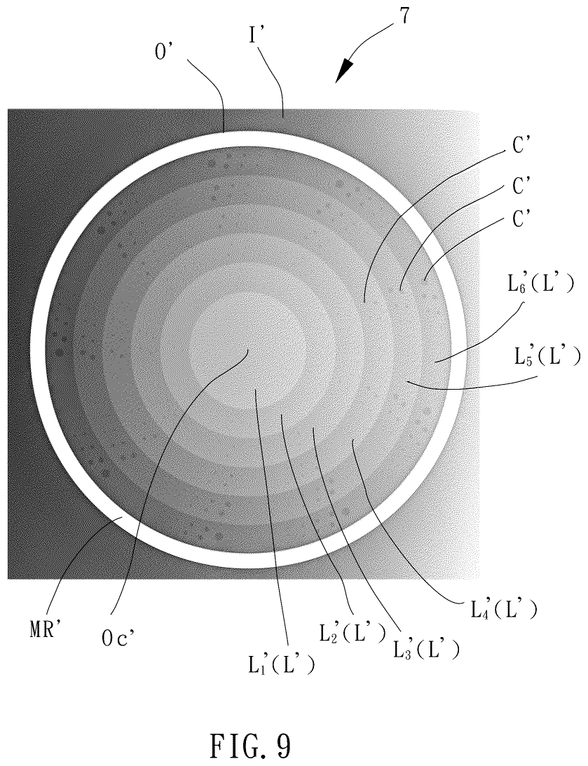

Please refer to FIG. 9, which shows an image I obtained by irradiating the test phantom 1 (corresponding to the example in FIG. 7) with electromagnetic radiation (X-ray). The image I contains an image of the test phantom 1′, preferably with images of the layer bodies L_{1′} to L_{N′} corresponding to the layer bodies L₁ to L_{N}, and more preferably with images of the recess portions C′ corresponding to the recess portions C, and ideally with an image of the metal ring MR′ corresponding to the metal ring MR. It should be noted that the various feature images shown in FIG. 9 (the images of the layer bodies L_{1′} to L_{N′}, the images of the recess portions C′, and the image of the metal ring MR′) will either fully or partially represent the images of each feature in the test phantom 1, depending on the quality of the actual electromagnetic radiation. Optionally, a center Oc′ of the image of the test phantom 1′ can be defined in a contour O′ associated with the center of the test phantom 1, and corresponding two-dimensional coordinates can be defined.

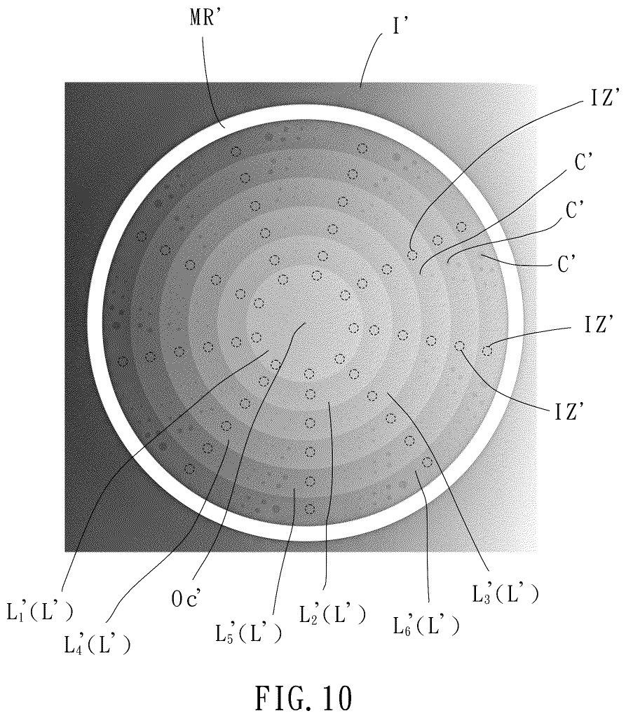

Please refer to FIG. 10, which shows a schematic diagram of a method for evaluating image uniformity. Specifically, to calculate the uniformity of the image, using the center Oc′ as the reference point, a zone of interest IZ′ corresponding to each image of the layer body L′ within each of the first subarea 11 is selected, and the mutual information values of each zone of interest IZ′ for each image of the layer body L′ are calculated to assess the image uniformity of each image of the layer body L′ around the center Oc′. Especially, the maximum value of the mutual information calculated in each image of the layer body L′ will be significantly influenced by the number of layers. Therefore, in order to facilitate the observation of the uniformity of the same layer at different angles, each of the multiple mutual information values calculated for each layer will be divided by the maximum value among these mutual information values, to obtain a normalized mutual information value. It should be noted that the mutual information values are obtained by calculating the Shannon entropy, which can be understood by a person having ordinary skill in the art, and thus detailed descriptions are omitted.

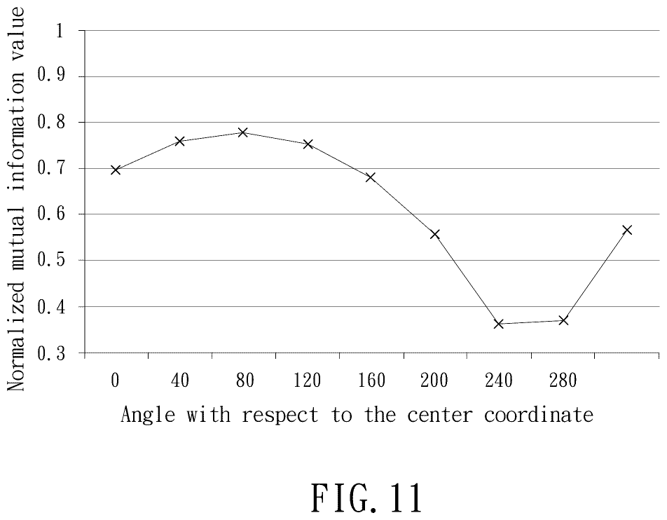

Referring to FIG. 11, based on the example in FIG. 10, where the specific angle is 40 degrees and 9 test areas 10 are defined, it shows the normalized mutual information values for each image of the layer body L′ corresponding to each first subarea 11 at the respective angle. The higher the mutual information value at the corresponding angle, the higher the color correlation around the zone of interest IZ′, indicating a higher degree of uniformity. In addition, the range with the highest uniformity can be used to find the angle range where the electromagnetic radiation is more uniform (corresponding to the cathode of the X-ray), while the range with the lowest uniformity can be used to identify the angle range where the intensity of the electromagnetic radiation is less uniform (corresponding to the anode of the X-ray).

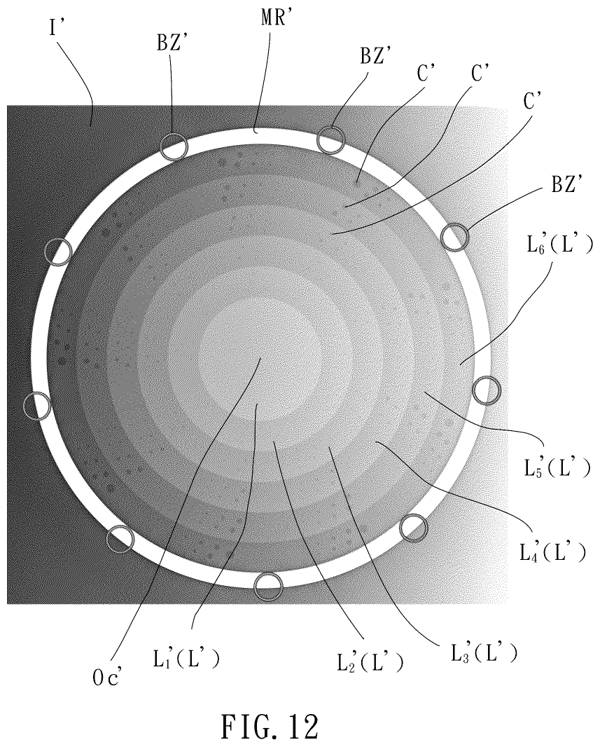

Please refer to FIG. 12, which shows a schematic diagram for evaluating the contrast resolution of the image, and the contrast resolution is assessed using the image of the metal ring MR′. In detail, to calculate the contrast resolution, a boundary zone BZ′ is defined at the interface between the image of the metal ring MR′ and the background area (the part outside the image of the test phantom 1′ and the image of the metal ring MR′) within the obtained image. A plurality of boundary zones BZ′ is then defined at various angular directions with reference to the center Oc′. From each boundary zone BZ′, the edge spread function (ESF) is obtained. The ESF is then processed through a standard calculation procedure involving the Fourier transform to obtain the modulation transfer function (MTF). In particular, since the metal ring MR uniformly surrounds the outer periphery of the test phantom 1, the MTF can be calculated for various angular directions. By doing so, the full width at half maximum (FWHM) of the MTF can be assessed to observe the contrast resolution in different angular directions. It should be noted that the calculations of the ESF and the MTF can be understood by a person having ordinary skill in the art, and detailed descriptions are thus omitted.

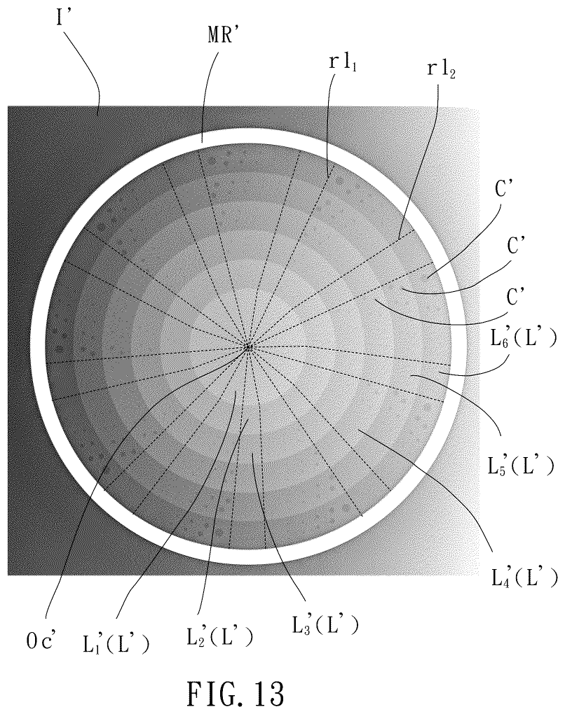

Please refer to FIG. 13, which shows a schematic diagram for evaluating the detail resolution of the image, and the detail resolution is assessed using the image of the recess portion C′ of each layer body L′. For each corresponding second subarea 12, the contrast-to-noise ratio (CNR) can be calculated using the predetermined imaging coordinate center point of each image of the recess portion C′ and the surrounding background image. When the CNR exceeds a predetermined threshold, the image of the recess portion C′ is deemed recognizable and is counted as a detected image of the recess portion C′. By dividing the number of the detected images of the recess portions C′ by the total number of the recess portions C in each corresponding second subarea 12, the detail detection rate for each second subarea 12 at the specific angle can be obtained. This enables the assessment of detail resolution and image quality uniformity across different angular orientations.

From the above descriptions, it can be understood that the mutual information values calculated from each first area 11 of each image of the layer body L′ corresponding to each layer body L can be used to assess the uniformity of the images. The MTF calculated based on the image of the metal ring MR′ corresponding to the metal ring MR and the background area can be used to assess the contrast resolution of the image. The detail detection rate, calculated from each image of the recess portion C′ corresponding to each recess portion C in the respective second subarea 12, can be used to assess the detail resolution of the images.

Optionally, based on the relationship between the positions and proportions of distinctive features between the aforementioned test phantom 1 and the corresponding image of the test phantom 1′, alignment features can be specifically designed to appear in the image of the test phantom 1′ in order to confirm the orientation of the test phantom 1. Furthermore, a computer equipped with a corresponding image recognition model can be utilized to identify the image of the layer body L_{1′} to L_{N′}, the image of the recess portion C′, and the image of the metal ring MR′ within the phantom image 1′. Subsequently, utilizing algorithms corresponding to image quality assessment indicators, automated calculation and evaluation of image uniformity, contrast resolution, and detail resolution can be achieved.

It should be noted that the zones of interest IZ′ distributed in various orientations in FIG. 10, the boundary zones BZ′ distributed in various orientations in FIG. 12, and the first and second reference lines rl₁ and rl₂ in FIG. 13 are merely auxiliary indications for the purpose of clearly illustrating the calculation process and are not intended to limit the manner in which the present invention calculates various image indicators.

In summary, the test phantom for evaluating electromagnetic radiation images according to the present invention enables the calculation of detail detection rates through various recess portions, thereby permitting the assessment of the detail resolution of the images. Additionally, by arranging the recess portions in different layer bodies, even if the recess portions all have the same depth, corresponding attenuation of electromagnetic radiation can still be achieved based on the different layer bodies, thereby eliminating the need for differentiation in depth among the recess portions and simplifying the corresponding manufacturing process. Furthermore, through the arrangement of the various layer bodies, particularly with each feature configured symmetrically around a centroid or distributed in a rotational array, and preferably by capturing images of the planar portions of the layer bodies in various orientations, mutual information values can be calculated to assess the uniformity of the images. Moreover, through the configuration of the metal ring, the modulation transfer function can be calculated to evaluate the contrast resolution of the image. In addition, through the configuration of the aforementioned various corresponding features, multiple assessments of image quality can be realized.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims.

What is claimed is:

1. A test phantom for evaluating electromagnetic radiation images, comprising:
a plurality of layer bodies stacked one upon another to form stacked layer bodies, wherein an area of each of the plurality of layer bodies gradually increases from top to bottom of the stacked layer bodies, and each of the plurality of layer bodies has an exposed region on an upper surface thereof; and;
a plurality of recess portions formed by recessing the exposed regions of the upper surfaces of at least two of the plurality of layer bodies, wherein the recess portions located on different layer bodies cause different degrees of attenuation to electromagnetic radiation passing therethrough.

2. The test phantom for evaluating electromagnetic radiation images as claimed in claim 1, wherein the test phantom has a centroid in a viewing direction, and the plurality of recess portions is symmetrically arranged or is distributed in a ring array around the centroid in the viewing direction.

3. The test phantom for evaluating electromagnetic radiation images as claimed in claim 1, wherein after stacking, each of the plurality of layer bodies renders an outline of the test phantom to be symmetrically arranged or to be distributed in a ring array around a centroid of the test phantom in a viewing direction.

4. The test phantom for evaluating electromagnetic radiation images as claimed in claim 1, wherein in each two adjacent layer bodies, with each of which having at least one of the plurality of recess portions, a radial length of one or more of the at least one of the plurality of recess portions in a lower one of the two adjacent layer bodies is not less than a radial length of any of the at least one of the plurality of recess portions in an upper one of the two adjacent layer bodies.

5. The test phantom for evaluating electromagnetic radiation images as claimed in claim 1, wherein in each two adjacent layer bodies, with each of which having at least one of the plurality of recess portions, a radial length of one or more of the at least one of the plurality of recess portions in a lower one of the two adjacent layer bodies is greater than a radial length of any of the at least one of the plurality of recess portions in an upper one of the two adjacent layer bodies.

6. The test phantom for evaluating electromagnetic radiation images as claimed in claim 1, wherein each of the plurality of recess portions is a through-hole.

7. The test phantom for evaluating electromagnetic radiation images as claimed in claim 1, wherein one of the plurality of layer bodies has a plurality of recess portions with different radial lengths, and the radial lengths are arranged according to the Fibonacci sequence.

8. The test phantom for evaluating electromagnetic radiation images as claimed in claim 1, wherein a bottommost layer body in the plurality of layer bodies has a metal ring disposed around an outer periphery thereof.
